(19) United States
(12) Patent Application Publication
(10) Pub. No.: US 2007/0156190 A1
(43) Pub. Date: Jul. 5, 2007

(54) Subcutaneous ICD with motion artifact noise suppression

(76) Inventor: Can Cinbis, Shoreview, MN (US)

Correspondence Address: MEDTRONIC, INC., 710 MEDTRONIC PARKWAY NE, MINNEAPOLIS, MN, 55432-9924, US

(21) Appl. No.: 11/324,076

(22) Filed: Dec. 30, 2005

(51) Int. Cl. A61N 1/00

(52) U.S. Cl. 607/5

(57) ABSTRACT

A subcutaneous implantable cardioverter defibrillator (SubQ ICD) includes a housing carrying electrodes for sensing ECG signals and delivering therapy. A sensor detects local motion in the area of the housing and produces a noise signal related to motion artifact noise contained in ECG signals derived from the electrode array. An adaptive noise cancellation circuit enhances ECG signals based on the local motion noise signal. A therapy delivery circuit delivers cardioversion and defibrillation pulses based upon the enhanced ECG signals.

### BACKGROUND OF THE INVENTION

The present invention relates to implantable medical devices. In particular, the invention relates to a subcutaneous implantable cardioverter defibrillator (SubQ ICD) in which motion artifact noise associated with local motion near the SubQ ICD is sensed and used to enhance sensed subcutaneous ECG signals.

Implantable cardioverter defibrillators are used to deliver high energy cardioversion or defibrillation shocks to a patient's heart when atrial or ventricular fibrillation is detected. Cardioversion shocks are typically delivered in synchrony with a detected R-wave when fibrillation detection criteria are met. Defibrillation shocks are typically delivered when fibrillation criteria are met, and the R-wave cannot be discerned from signals sensed by the ICD.

Currently, ICDs use endocardial or epicardial leads which extend from the ICD housing to the heart. The housing generally is used as an active can electrode for defibrillation, while electrodes positioned in or on the heart at the distal end of the leads are used for sensing and delivering therapy.

The SubQ ICD differs from the more commonly used ICDs in that the housing is typically smaller and is implanted subcutaneously. The SubQ ICD does not require leads to be placed in the bloodstream. Instead, the SubQ ICD makes use of one or more electrodes on the housing, together with a subcutaneous lead that carries a defibrillation coil electrode and a sensing electrode.

The lack of endocardial or epicardial electrodes make sensing more challenging with the SubQ ICD. Sensing of atrial activation is limited since the atria represent a small muscle mass, and the atrial signals are not sufficiently detectable thoracically. Muscle movement, respiration, and other physiological signal sources also can affect the ability to sense ECG signals and detect arrhythmias with a SubQ ICD.

### BRIEF SUMMARY OF THE INVENTION

A SubQ ICD includes a local motion sensor for producing a signal related to motion artifact noise contained in ECG signals derived by an electrode array carried on the SubQ ICD housing. An adaptive noise cancellation circuit enhances ECG signals derived from the electrode array based on the signal from the local motion sensor. The enhanced ECG signals are used for arrhythmia detection and delivery of therapy.

### BRIEF DESCRIPTION OF THE DRAWINGS

FIG. 1 depicts a SubQ ICD implanted in a patient.

FIGS. 2A and 2B are front and top views of the SubQ ICD associated electrical lead shown in FIG. 1.

FIG. 3 is a circuit diagram of circuitry of the SubQ ICD.

FIG. 4 is a block diagram of sensing circuitry of the SubQ ICD, including an adaptive noise cancellation circuit.

### DETAILED DESCRIPTION

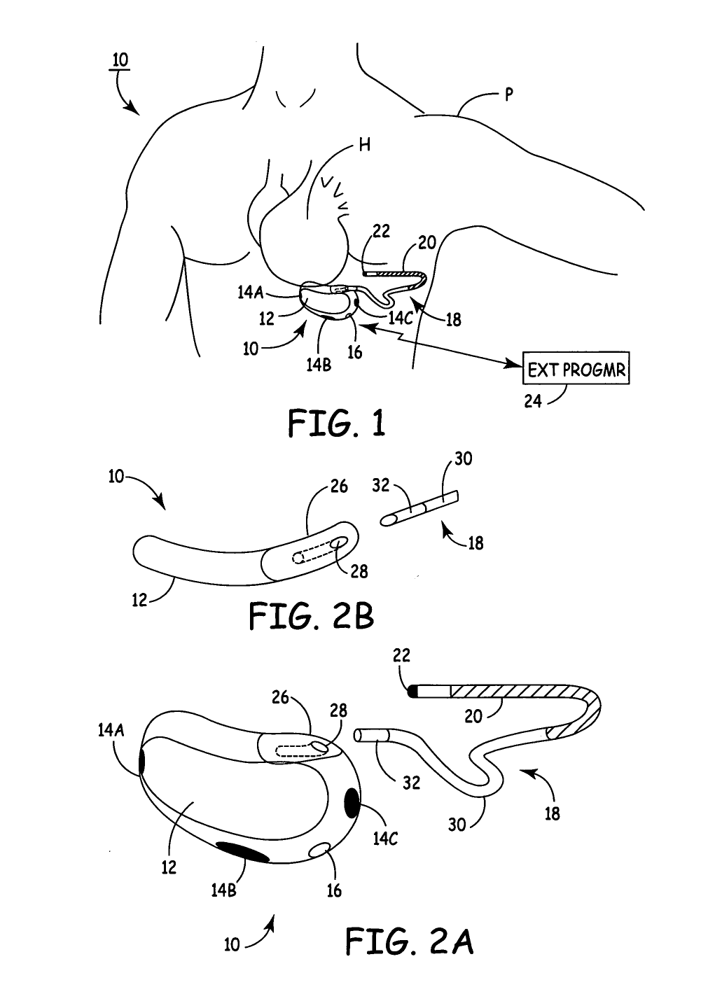

FIG. 1 shows SubQ ICD 10 implanted in patient P.

Housing or canister 12 of SubQ ICD 10 is subcutaneously implanted outside the ribcage of patient P, anterior to the cardiac notch, and carries three subcutaneous electrodes 14A-14C and local motion sensor 16.

Subcutaneous sensing and cardioversion/defibrillation therapy delivery lead 18 extends from housing 12 and is tunneled subcutaneously laterally and posterially to the patient's back at a location adjacent to a portion of a latissimus dorsi muscle. Heart H is disposed between the SubQ ICD housing 12 and distal electrode coil 20 of lead 18. SubQ ICD 10 communicates with external programmer 24 by an RF communication link.

FIGS. 2A and 2B are front and top views of SubQ ICD 10.

Housing 12 is an ovoid with a substantially kidney-shaped profile. The ovoid shape of housing 12 promotes ease of subcutaneous implant and minimizes patient discomfort during normal body movement and flexing of the thoracic musculature. Housing 12 contains the electronic circuitry of SubQ ICD 10. Header 26 and connector 28 provide an electrical connection between distal electrode coil 20 and distal sensing electrode 22 on lead 18 and the circuitry with housing 12.

Subcutaneous lead 18 includes distal defibrillation coil electrode 20, distal sensing electrode 22, insulated flexible lead body 30 and proximal connector pin 32. Distal sensing electrode 22 is sized appropriately to match the sensing impedance of electrodes 14A-14C.

Electrodes 14A-14C are welded into place on the flattened periphery of canister 12 and are connected to electronic circuitry inside canister 12. Electrodes 14A-14C may be constructed of flat plates, or alternatively, spiral electrodes as described in U.S. Pat. No. 6,512,940 entitled “Subcutaneous Spiral Electrode for Sensing Electrical Signals of the Heart” to Brabec, et al. and mounted in a non-conductive surround shroud as described in U.S. Pat. Nos. 6,522,915 entitled “Surround Shroud Connector and Electrode Housings for a Subcutaneous Electrode Array and Leadless ECGs” to Ceballos, et al. and 6,622,046 entitled “Subcutaneous Sensing Feedthrough/Electrode Assembly” to Fraley, et al. Electrodes 14A-14C shown in FIG. 2 are positioned on housing 12 to form orthogonal signal vectors.

Local motion sensor 16 is a pressure sensor, optical sensor, impedance sensor or accelerometer positioned to detect motion in the vicinity of electrodes 14A-14C, which are susceptible to motion artifact noise in the ECG signals. As shown in FIG. 2A, local motion sensor 16 is mounted on the exterior of canister 12, but is may also be mounted interiorly, so long as it can detect motion in the vicinity of electrodes 14A-14C. Specificity and sensitivity of a signal detection algorithm for electrodes 14A-14C is likely to suffer for a SubQ ICD device due to electrode distance from the heart and the proximity of large muscles in the chest. Local motion sensor 16 provides a way of improving specificity of the detection algorithm. Detection of reliable ECG signals is an essential requirement for proper operation of an implantable device such as an ICD or an external defibrillator. For a device that has no endocardial or epicardial leads, as its electrodes get farther away from the heart, ECG signal strength will degrade. Under these conditions, detection circuitry may be more prone to false detects. Noise due to muscle motion in the vicinity of ECG sensing electrodes may cause spurious electrical signals that could be interpreted as QRS complexes by the detection circuitry and algorithm. This might lead to delivery of unnecessary shocks or a necessary shock being held off, causing adverse outcomes for the patient. However, by using local motion detector 16 in the vicinity of electrodes 14A-14C, a signal representative of the motion that causes motion artifacts in the ECG signals can be acquired. By employing adaptive noise cancellation algorithms, this local motion signal can be used as correlated noise to eliminate motion generated noise present in the ECG channel.

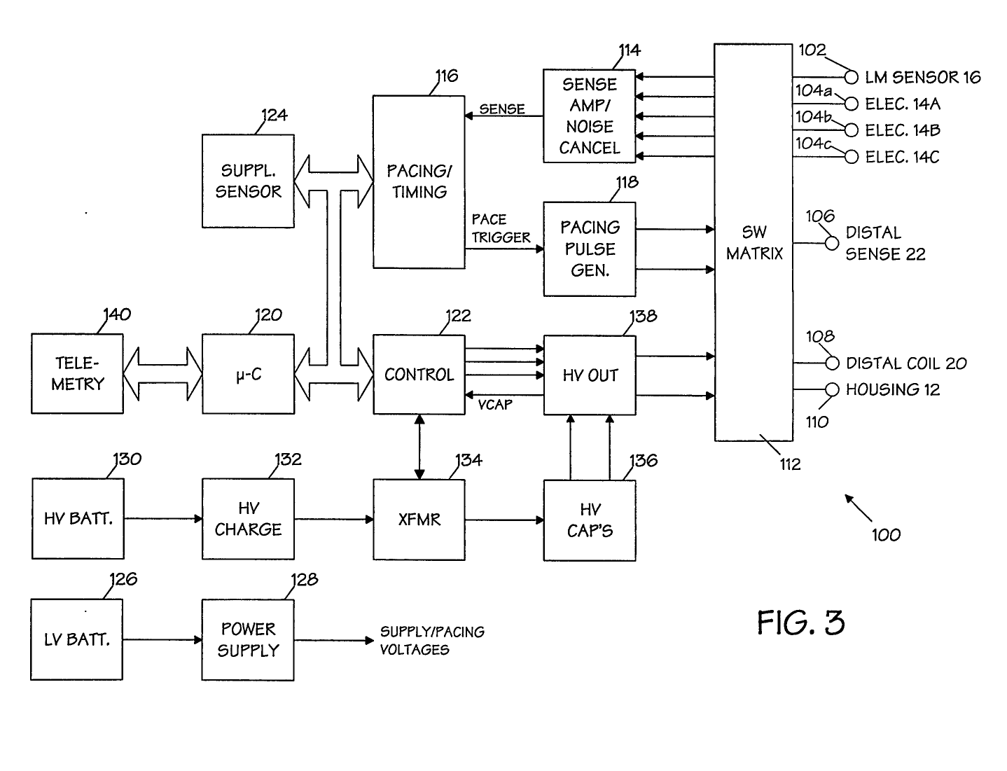

FIG. 3 is a block diagram of electronic circuitry 100 of SubQ ICD 10. Circuitry 100, which is located within housing 12, includes terminals 102, 104A-104C, 106, 108 and 110; switch matrix 112; sense amplifier/noise cancellation circuitry 114; pacer/device timing circuit 116; pacing pulse generator 118; microcomputer 120; control 122; supplemental sensor 124; low-voltage battery 126; power supply 128; high-voltage battery 130; high-voltage charging circuit 132; transformer 134; high-voltage capacitors 136; high-voltage output circuit 138; and telemetry circuit 140.

Terminal 102 is connected to local motion sensor 16 for receipt of a local motion signal input. Switch matrix 112 provides the local motion signal by sensing amplifier/noise cancellation circuit 114 for use as correlated noise to eliminate motion artifact noise in ECG input signals.

Electrodes 14A-14C are connected to terminals 104A-104C. Electrodes 14A-14C act as both sensing electrodes to supply ECG input signals through switch matrix 112 to sense amplifier/noise cancellation circuit 114, and also as pacing electrodes to deliver pacing pulses from pacing pulse generator 118 through switch matrix 112.

Terminal 106 is connected to distal sense electrode 22 of subcutaneous lead 18. The ECG signal sensed by distal sense electrode 22 is routed from terminal 106 through switch matrix 112 to sense amplifier/noise cancellation circuit 114.

Terminals 108 and 110 are used to supply a high-voltage cardioversion or defibrillation shock from high-voltage output circuit 138.

Terminal 108 is connected to distal coil electrode 20 of subcutaneous lead 18. Terminal 110 is connected to housing 12, which acts as a common or can electrode for cardioversion/defibrillation.

Sense amplifier/noise cancellation circuit 114 and pacer/device timing circuit 116 process the ECG signals from electrodes 14A-14C and 22, and the local motion signal from local motion sensor 16. Signal processing is based upon the transthoracic ECG signal from distal sense electrode 22 and a housing-based ECG signal received across an ECG sense vector defined by a selected pair of electrodes 14A-14C, or a virtual vector based upon signals from all three sensors 14A-14C. Both the transthoracic ECG signal and the housing-based ECG signal are amplified and bandpass filtered by preamplifiers, sampled and digitized by analog-to-digital converters, and stored in temporary buffers. In the case of the housing-based ECG signal, adaptive filtering is also performed using the local motion signal from sensor 16 to remove noise caused by local motion artifacts.

Bradycardia is determined by pacer/device timing circuit 116 based upon R waves sensed by sense amplifier/noise cancellation circuit 114. An escape interval timer within pacer/device timing circuit 116 or control 122 establishes an escape interval. Pace trigger signals are applied by pacer/device timing circuit 116 to pacing pulse generator 118 when the interval between successive R waves sensed is greater than the escape interval.

Detection of malignant tachyarrhythmia is determined in control circuit 122 as a function of the intervals between R wave sense event signals from pacer/device timing circuit 116. This detection also makes use of signals from supplemental sensor(s) 124 as well as additional signal processing based upon the ECG input signals.

Supplemental sensor(s) 124 may sense tissue color, tissue oxygenation, respiration, patient activity, or other parameters that can contribute to a decision to apply or withhold defibrillation therapy.

Supplemental sensor(s) 124 can be located within housing 12, or may be located externally and carried by a lead to switch matrix 112.

Microcomputer 120 includes a microprocessor, RAM and ROM storage and associated control and timing circuitry. Detection criteria used for tachycardia detection may be downloaded from external programmer 24 through telemetry interface 140 and stored by microcomputer 120.

Low-voltage battery 126 and power supply 128 supply power to circuitry 100. In addition, power supply 128 charges the pacing output capacitors within pacing pulse generator 118. Low-voltage battery 126 can comprise one or two LiCFₓ, LiMnO₂ or Lil₂ cells.

High-voltage required for cardioversion and defibrillation shocks is provided by high-voltage battery 130, high-voltage charging circuit 132, transformer 134, and high-voltage capacitors 136. High-voltage battery 130 can comprise one or two conventional LiSVO or LiMnO₂ cells.

When a malignant tachycardia is detected, high-voltage capacitors 136 are charged to a preprogrammed voltage level by charging circuit 132 based upon control signals from control circuit 122.

Feedback signal Vcap from output circuit 138 allows control circuit 122 to determine when high-voltage capacitors 136 are charged. If the tachycardia persists, control signals from control 122 to high-voltage output signal 138 cause high-voltage capacitors 136 to be discharged through the body and heart H between distal coil electrode 20 and the can electrode formed by housing 12.

Telemetry interface circuit 140 allows SubQ ICD 10 to be programmed by external programmer 24 through a two-way telemetry link. Uplink telemetry allows device status and other diagnostic/event data to be sent to external programmer 24 and reviewed by the patient's physician. Downlink telemetry allows external programmer 24, under physician control, to program device functions and set detection and therapy parameters for a specific patient.

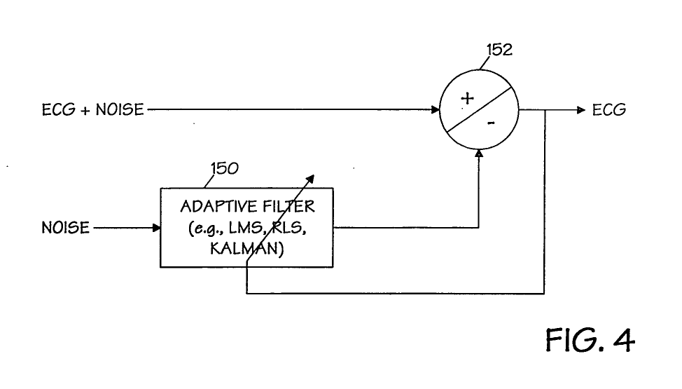

FIG. 4 is a block diagram showing noise cancellation algorithm used by sense amplifier/noise cancellation circuit 114. FIG. 4 illustrates a signal (ECG+Noise), which is received from one or more of electrodes 14A-14C. An additional input is a Noise signal produced by local motion sensor 16. The Noise signal from sensor 16 is processed by adaptive filter 150 and is subtracted at summing junction 152 from the ECG+Noise signal derived from electrodes 14A-14C. The output of summing junction 152 is an enhanced ECG signal with some or all of the motion artifact noise removed. This enhanced ECG signal is used as a feedback signal to adaptive filter 150 to control the subtraction signal supplied to junction 152.

Adaptive filter 150 can use adaptive filtering algorithms based on Least Means Squared (LMS), Recursive Least Squares (RLS) or Kalman filtering methods, or other methods such as multiplication free algorithms that increase computational efficiency and reduce power consumption.

In order to conserve energy, sense amplifier/noise cancellation circuit 114 may selectively use the noise cancellation feature depending upon the content of the input ECG signals. This can be achieved, for example, by monitoring RMS (Root Mean Square) power of the local motion sensor signal and performing noise cancellation only when the power exceeds a threshold level.

In another embodiment, the spectrum of the ECG input signals can be analyzed to determine when noise cancellation is appropriate. The ECG signal typically has a narrow band spectrum, which will widen with the presence of noise. Upon detecting spectrum broadening of the ECG signal, the noise cancellation feature is initiated.

Although a single local motion sensor 16 has been shown and discussed, multiple local motion sensors can be used, with the Noise signal used for cancellation being derived from one or a combination of the motion sensor signals. The motion sensor can be a pressure sensor, an optical sensor, an impedance sensor or an accelerometer.

For example, an optical sensor used for local motion sensing may include a light emitting diode radiating at an isobestic wavelength for oxygen (such as 810 nm or 569 nm), so that it has no sensitivity to local oxygen change, and a photodetector to collect light scattered by local tissue. Motion will cause changes in tissue optical density, and the amount of light collected by the photodetector will be modulated by motion.

A local motion sensor using pressure sensing can make use of a piezoresistive, piezoelectric or capacitive sensor located in the housing. Pressure exerted on the surrounding tissue by housing 12 produces a pressure sensor output representing local motion.

An impedance sensor sharing one or more of ECG electrodes or dedicated electrodes can be used to measure local tissue impedance. Changes in the electrode-electrolyte (tissue) interface due to motion artifacts can be sensed via changes in the magnitude and/or phase of the local impedance signal. Impedance measurement can be performed via narrowband sinusoidal excitation outside of the ECG bandwidth so as not interfere with ECG sensing.

An accelerometer may also be used to sense motion of housing 12 and electrodes 14. However, an accelerometer will sense motion globally, and may sometimes detect motion that does not affect the ECG signal. Depending upon the activity of the patient, and other sensor signals that may be used in conjunction with the accelerometer signal, an accelerometer may provide a sufficiently accurate correlation to local motion to permit noise cancellation of the ECG signals.

Although the present invention has been described with reference to preferred embodiments, workers skilled in the art will recognize that changes may be made in form and detail without departing from the spirit and scope of the invention.

What is claimed is:

1. A subcutaneous ICD comprising:
an ICD housing;
a therapy delivery lead carrying a defibrillation electrode;
an electrode array carried on an exterior of the ICD housing;
sensing circuitry within the ICD housing connected to the electrode array for producing ECG signals;
a local motion sensor for producing a noise signal related to motion artifact noise contained in the ECG signals;
an adaptive noise cancellation circuit for enhancing the ECG signals based on the noise signal; and
therapy delivery circuitry within the ICD housing connected to the defibrillation electrode for providing electrical pulses to the defibrillation electrode upon detection of tachycardia based on the enhanced ECG signals.

2. The subcutaneous ICD of claim 1, wherein the electrode array includes first, second, and third electrodes.

3. The subcutaneous ICD of claim 1, wherein the local motion sensor is carried by the housing.

4. The subcutaneous ICD of claim 1, wherein the local motion sensor comprises an optical sensor.

5. The subcutaneous ICD of claim 1, wherein the local motion sensor comprises a pressure sensor.

6. The subcutaneous ICD of claim 1, wherein the local motion sensor comprises an impedance sensor.

7. The subcutaneous ICD of claim 1, wherein the local motion sensor comprises an accelerometer.

8. The subcutaneous ICD of claim 1, wherein the adaptive noise cancellation circuit performs noise cancellation as a function of detected power of the noise signal.

9. The subcutaneous ICD of claim 1, wherein the adaptive noise cancellation circuit performs noise cancellation as a function of a spectral bandwidth of the ECG signals.

10. The subcutaneous ICD of claim 1, wherein the noise cancellation circuit performs noise cancellation based on at least one of Least Mean Squares filtering, Recursive Least Squares filtering, Kalman filtering and multiplication-free adaptive filtering.

11. A method of providing therapy with a subcutaneous ICD, the method comprising:
sensing ECG signals with a plurality of electrodes carried by a housing of the subcutaneous ICD;
sensing local motion associated with relative movement of the housing and adjacent tissue;
performing adaptive noise cancellation of the ECG signals as a function of the sensed local motion;
detecting tachycardia based upon the ECG signals; and
delivering an electrical pulse in response to detected tachycardia.

12. The method of claim 11, wherein a local motion sensor carried by the housing senses local motion.

13. The method of claim 12, wherein the local motion sensor comprises at least on of an optical sensor, a pressure sensor, and an impedance sensor.

14. The method of claim 12, wherein the local motion sensor comprises an accelerometer.

15. The method of claim 11, wherein the adaptive noise cancellation is performed as a function of detected power of the noise signal.

16. The method of claim 11, wherein the adaptive noise cancellation is performed as a function of a spectral bandwidth of the ECG signals.

17. The method of claim 1, wherein the adaptive noise cancellation includes at least one of Least Mean Squares filtering, Recursive Least Squares filtering, Kalman filtering and multiplication-free adaptive filtering.

18. A subcutaneous implantable medical device comprising:
an ICD housing configured for subcutaneous implantation;
an electrode array carried on an exterior of the housing;
sensing circuitry within the housing connected to the electrode array for producing ECG signals;
a local motion sensor for producing a noise signal related to relative motion of the housing and surrounding tissue;
an adaptive noise cancellation circuit for removing motion artifact noise from the ECG signals as a function of the noise signal.

19. The subcutaneous implantable medical device of claim 18, and further comprising:
therapy delivery circuitry within the housing for providing electrical therapy based on the ECG signals.

20. The subcutaneous implantable medical device of claim 18, wherein the local motion sensor comprises at least one of an optical sensor, a pressure sensor, an impedance sensor and an accelerometer.
